(19) United States
(12) Patent Application Publication
(10) Pub. No.: US 2011/0076186 A1
(43) Pub. Date: Mar. 31, 2011

(54) METHOD AND APPARATUS FOR TREATING AIR

(76) Inventors: David Itzhak, Tel-Aviv (IL); Modiin Niv, Modiin (IL)

(21) Appl. No.: 12/736,009

(22) PCT Filed: Mar. 8, 2009
(86) PCT No.: PCT/IL09/00253

§ 371 Date: Nov. 15, 2010

Related U.S. Application Data
(60) Provisional application No. 61/064,452, filed on Mar. 6, 2008.

(51) Int. Cl. A61L 9/00; A62B 7/00

(52) U.S. Cl. 422/4; 422/122

(57) ABSTRACT

The invention provides an apparatus and method for treating indoor air, utilizing a container suitable for holding a brine solution thereinside, a porous medium rotatably mounted on, or in, said container such that a section thereof is capable of being immersed in the brine solution held inside it, rotating means mechanically coupled to said porous medium, and air streaming means capable of directing a stream of air to a surface area of said porous medium.

### FIELD OF THE INVENTION

The present invention relates to a method and an apparatus for purifying and humidifying air. More particularly, the invention relates to a method and apparatus for purifying and humidifying contaminated air by contacting a stream of ambient air with a brine solution.

### BACKGROUND OF THE INVENTION

Commonly used methods for treating contaminated air are based on filtration, air ionization and sterilization of air by means of ozone or ultraviolet light.

WO 2007/026363 describes a method for reducing the level of microorganism of indoor air, by contacting a stream of indoor air with a concentrated salt solution, which is preferably halide brine having a Redox (reduction-oxidation) potential in the range between 200 mV and 450 mV. The publication identifies certain brines that are capable of developing such Redox potentials under suitable aeration conditions, and alternatively or in addition, proposes to electrolyze the brine in an electrolytic cell, whereby the Redox potential of the brine is suitably adjusted. WO 2007/026363 also specifically describes a packed column scrubber (in which the air and the brine solution are brought into contact via solid material filling the column) for running the method set forth above in various facilities, including hospitals.

It has been demonstrated that operating under the preferred treatment regime according to WO 2007/026363, using brines having Redox potentials in the range between 200 and about 450 mV, effectively reduces the level of biological contaminants of indoor air. There exists a need, however, in certain facilities, to periodically sanitize the air, the walls and other surfaces in closed spaces.

There is still a need for cost effective solutions for purifying air and controlling its humidity in houses and relatively small spaces.

It is therefore an object of the present invention to provide a simplified method and apparatus for purifying air inside houses and relatively small spaces.

It is another object of the present invention to provide a method and apparatus for purifying air and adjusting its humidity.

Other objects and advantages of the invention will become apparent as the description proceeds.

### SUMMARY OF THE INVENTION

The present invention provides a method and apparatus for treating air by contacting a stream of air with a brine solution contained in a revolving porous medium. In particular, the invention provides a method and apparatus for purifying contaminated air, and optionally also humidifying the purified air, by contacting a stream of ambient air with a brine solution contained in a revolving porous medium.

In a preferred embodiment of the invention a portion of the porous revolving medium is maintained immersed in a brine solution such that portions of the brine solution are continuously absorbed in its pores and moved thereinside to a treatment zone wherein a stream of ambient air is caused to pass through portions of the porous rotating medium maintained external to (not immersed in) the brine solution. Advantageously, the humidity of the air passed through the porous revolving medium may be controllably altered by adjusting the vapor pressure of the brine solution e.g., by means of a heating element such as a filament, in order to prevent excess absorption of moisture from the environment. Advantageously, whenever the ambient air is too dry moisture may be added to it (up to a level of about 60%) by increasing vapor pressure in order to improve the air conditions. For this purpose the invention further provides means for monitoring the humidity of the purified air, for monitoring and controlling the temperature of the brine solution and for monitoring and adjusting its quantity, concentration, and activity.

In one aspect the present invention is directed to an apparatus for purifying air comprising a rotatable porous drum (e.g., comprising interconnected pores) mounted for revolution within a container, wherein a section of said drum extends outside of said container, rotating means for rotating (e.g., electrical motor) the rotatable porous drum within the container, and air streaming means for directing a stream of air to a surface area of the section of rotatable porous drum extending outside of said container.

In operation, the container is filled with a brine solution, such that a portion of the revolving porous drum is immersed in the brine solution. While the porous drum is rotated about its axis, a stream of ambient air is forced to pass through the section of the drum in the treatment zone i.e., which extends outside of the brine solution. The pores of the revolving porous drum are repeatedly filled with fresh brine solution from the container, and the ambient air streamed therethrough contacts the brine solution within the porous drum, whereby the air is treated.

The apparatus may further comprise humidity sensing means adapted to contact the treated air and means for monitoring (e.g., a temperature sensor) and adjusting (e.g., a heating element) the temperature of a solution maintained inside the container responsive to indications received from said humidity sensing means and from temperature monitoring means installed inside the container.

The apparatus may further comprise level measuring means installed in the container, such that readings from said level measuring means may be used to control the level of the solution in the container. For example, whenever the solution level is too high the temperature of the solution may be increased in order to increase the vapor pressure and prevent moisture absorption from the environment.

In one preferred embodiment the apparatus further comprises a reservoir for holding water, wherein said reservoir is in fluid communication with said container, and means for controlling the flow of water (e.g., valve) from said reservoir to said container. Conveniently, the means for determining the level of the solution contained inside the container and the means for controlling the flow of water from said reservoir are used for adjusting the solution level in the container responsive to indications received from said level determining means.

The apparatus may further comprise means for measuring a Redox (Reduction-Oxidation) potential of the solution maintained inside the container and for adjusting its activity responsive to indications received from said Redox measuring means. For example, Redox potential may be increased by increasing the speed of the stream of air introduced into the treatment zone, and/or by increasing the speed of rotations of the rotatable porous drum; by means of an automated whisk or mixer device installed in the container and adapted to introduce bubbles into the solution; and/or by introducing air bubbles by means of an air pump.

The rotatable porous drum may optionally comprise one or more elongated members formed in, or attached on, one or more sides of the porous rotatable drum, wherein each of said elongated members comprises an elongated ditch adapted to collect brine solution in it, and an elongated slit provided in the base, and/or the sides, of said elongated ditch and adapted to discharge brine solution therefrom onto the surface of said porous drum.

In another aspect the present invention is also directed to a method for treating air, which method comprises rotating a porous drum in a brine solution and forcing a stream of air to pass through a section of said drum which extends from said brine solution into a treatment zone. Preferably, upon heating, the brine solution also serves for humidifying the treated air, in which case water is being fed into said brine solution to prevent possible crystallization of salts within the brine. The method further comprises periodically or continuously measuring the Redox potential of the brine and adjusting the Redox potential of said brine based on the measured value of the Redox potential by introducing Oxigen into the solution by one or more of the following means: increasing the rate of the stream of air introduced into the treatment zone; by increasing the speed of rotations of the rotatable porous drum; by means of an automated whisk or mixer device installed in the container and adapted to introduce air bubbles into the solution; and/or by introducing air bubbles into the solution by means of an air pump.

The term “brine solution”, as used herein, refers to concentrated solutions wherein the concentration of the salt dissolved therein is preferably not less than 10% (w/w), and preferably not less than 20% (w/w), and up to saturation at the relevant temperature. Compositionally, the concentrated salt solution operative in the present invention is an aqueous solution containing one or more water soluble salts represented by the formulas MX, M₂X and MX₂, wherein X is selected from the group consisting of chloride, bromide, iodide, sulfate and nitrate anions, and M indicates a metal cation, which is most preferably selected from the group consisting of lithium, sodium, potassium, calcium, magnesium and zinc, and mixtures thereof. Preferred brine solutions include concentrated solutions of sodium chloride and calcium chloride (with a concentration of not less than 20 wt %, and preferably not less than 30 wt %). Another preferred concentrated salt solution to be used according to the invention comprises a mixture of at least one bromide or iodide salt, in combination with at least one chloride salt of one or more of the following metals: Na⁺, K⁺, Mg²⁺ and Ca²⁺. An especially preferred solution contains a mixture of bromide and chloride salts dissolved therein in a total concentration of 30 to 40% by weight, with the cationic species being Mg²⁺, Ca²⁺, Na⁺ and K⁺. More specifically, the concentrations of the aforementioned ions are as follows: Mg²⁺: 30-50 g/liter; Ca²⁺: 10-20 g/liter; Na⁺: 30-50 g/liter; K⁺: 5-10 g/liter; Cl⁻: 150-240 g/liter; Br⁻: 3-10 g/liter. An example of such a solution is provided by the Dead Sea brine, which has the following typical (average) mineral composition: Mg²⁺: about 40.6 g/liter; Ca²⁺: about 16.8 g/liter; Na⁺: about 39.1 g/liter; K⁺: about 7.26 g/liter; Cl⁻: about 212.4 g/liter; Br⁻: about 5.12 g/liter, with the total concentration of salts dissolved therein being 33% by weight. Another preferred concentrated salt solution comprises a mixture of bromide and chloride salts dissolved in water in a total concentration of 30 to 40% by weight, with the cationic species being Mg²⁺, Ca²⁺, Na⁺ and K⁺, wherein the concentration of calcium chloride in said solution is effective in reducing the rate of evaporation of water therefrom, and is preferably in the range between 20 and 200 g/liter.

### BRIEF DESCRIPTION OF THE DRAWINGS

The present invention is illustrated by way of example in the accompanying drawings, in which similar references consistently indicate similar elements and in which:

FIG. 1 illustrates a longitudinal-section view of an air purifying apparatus demonstrating the basic principle of the invention;

FIG. 2 illustrates a longitudinal-section view of a preferred embodiment of the air purifying and humidifying apparatus of the invention;

FIG. 3 is a three dimensional perspective view of the longitudinal-section view shown in FIG. 2;

FIG. 4 is a three dimensional longitudinal-section view taken along line X-X in FIG. 2;

FIGS. 5A and 5B show an optional embodiment of a revolving drum comprising liquid collecting means, wherein FIG. 5A shows a perspective view of the revolving drum and FIG. 5B shows a perspective view of the optional liquid collecting means; and

FIG. 6 is a block diagram schematically illustrating electrical connectivity of a control unit in the apparatus of the invention.

It should be noted that the embodiments exemplified in the Figs. are not intended to be in scale and are in diagram form to facilitate ease of understanding and description.

### DETAILED DESCRIPTION OF PREFERRED EMBODIMENTS

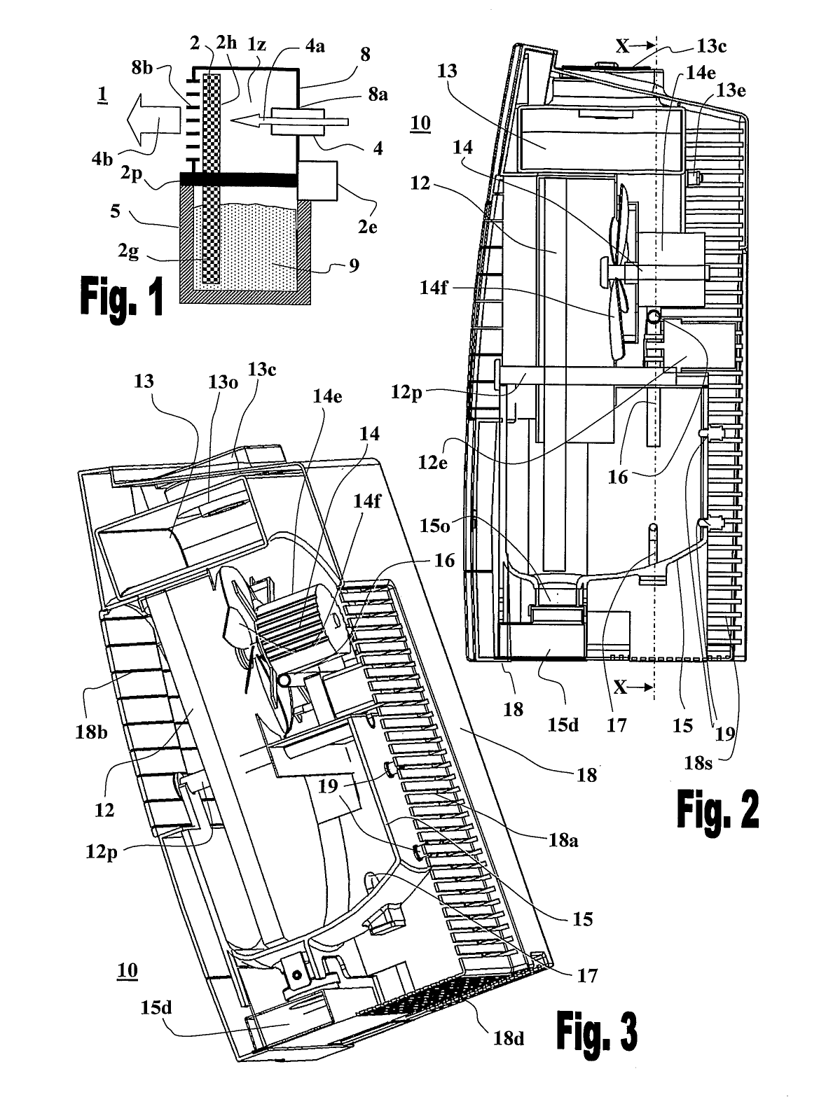

With reference to FIG. 1, air purifying and humidifying apparatus 1 of the invention is generally comprised of an open container 5 suitable for holding a volume of brine solution 9, a revolving porous drum 2 rotatably mounted on, or above, open container 5 by means of a rotatable shaft 2p, such that a portion 2g of revolving porous drum 2 is immersed inside brine solution 9 contained inside open container 5, rotating means 2e mechanically connected to rotatable shaft 2p, and air streaming means 4 capable of directing a stream of ambient air 4a onto a surface area 2h of revolving porous drum 2 in the treatment zone 1z i.e., external to open container 5. Upper part of revolving porous drum 2 is preferably enclosed within a cover 8 adapted to fit over it with close proximity, while not contacting it, such that substantial portions of ambient air stream 4a are forced to pass through a portion of revolving porous drum 2 in the treatment zone 1z. Cover 8 should comprise a rear opening 8a facing surface area 2h of revolving porous drum 2 for introducing the stream of ambient air 4a into apparatus 1, and a front opening(s) 8b for discharging a stream of treated air 4b leaving apparatus 1.

FIGS. 2 to 4 show longitudinal-section side, and three-dimensional perspective, views of an air purifying and humidifying apparatus 10 according to a preferred embodiment of the invention. Apparatus 10 is generally comprised of an open container 15 made of chemically resistant material such as, but not limited to, stainless alloys (such as austenitic, ferritic and martensitic stainless steels, mild steel, carbon steel, titanium alloys, nickel-based super alloys and cobalt alloys) or suitable plastics (such as PVC, CPVC, polyethylene, polypropylene, polybutylene, PVDF, Teflon and polyester), configured for holding a volume (e.g., 10 to 15 liters) of brine solution (e.g., Dead Sea brine), a revolving porous drum 12 rotatably mounted on, or above, open container 15 by means of rotatable shaft 12p, such that a portion of revolving porous drum 12 is maintained inside open container 15. Electrical motor 12e (e.g., a 220V/400-900 mA engine), mechanically linked to rotatable shaft 12p (e.g., made from plastic or metallic material, such as, but not limited to, stainless steel), is employed for rotating (e.g., 2 to 6 RPM) revolving porous drum 12 inside apparatus 10. An electrical fan 14 (e.g., a 300 to 1200 WAT engine, such as QC 20 or QC 25 manufactured by Dynair), comprising electrical motor 14e and rotatable fan 14f, is used for directing a stream (e.g., 100 to 200 m³/hr) of ambient air onto a surface area of revolving porous drum 12 external to open container 15.

Apparatus 10 is enclosed in housing 18 adapted to fit over the portion of revolving porous drum 12 external to open container 15, such that significant portions of the ambient air streamed by fan 14 are forced to pass through revolving porous drum 12 (i.e., thereby housing 18 implements the functionality of cover 8 shown in FIG. 1). Housing 18 should comprise at least two air openings: i) air inlet opening(s) which may comprise rear opening(s) 18a, side opening(s) 18s, and/or bottom opening(s) 18d, for introducing ambient air streams into apparatus 10; and i) front opening(s) 18b for discharging treated air from the apparatus. In operation, open container 15 is filled with a brine solution and revolving porous drum 12 is rotated about rotatable shaft 12p by electrical motor 12e, such that its active surface area is continuously wetted with fresh brine solution. The portions of the streams of ambient air driven by fan 14 which pass through revolving porous drum 12 contact the brine solution filling its active surface, thereby discharging a stream of treated air from apparatus 10 due to the activity of the brine solution and due to removal of solid particles (e.g., dust, soot) and liquid droplets by the brine solution.

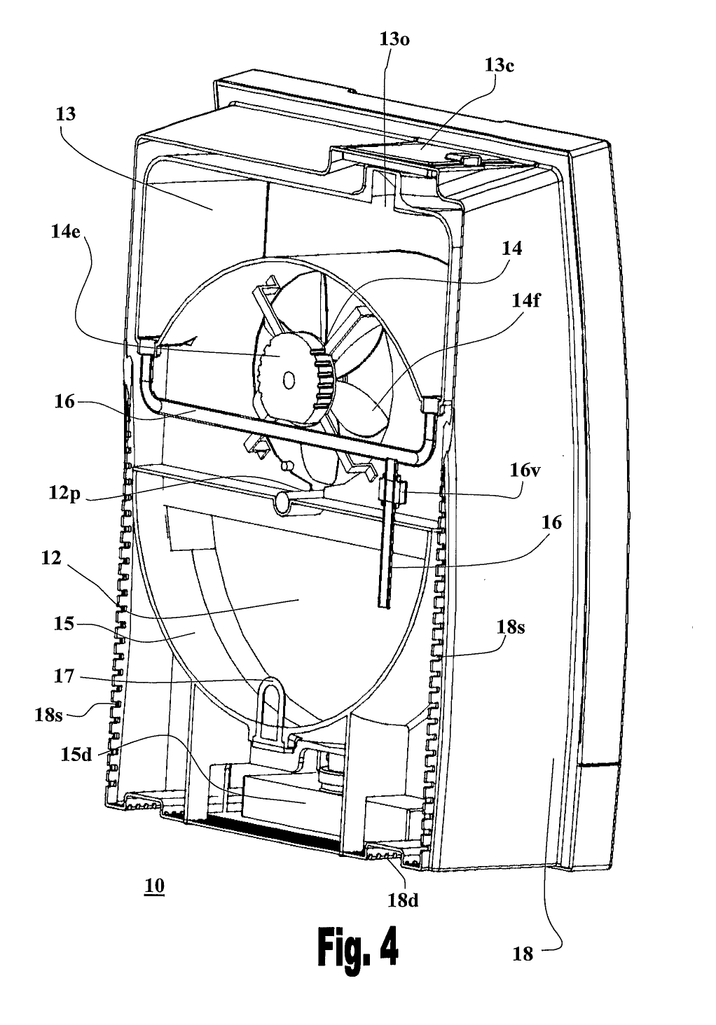

In one preferred embodiment of the invention apparatus 10 further comprises a water reservoir 13 installed inside housing 18 and adapted to fit over, and in close proximity to, the portion of revolving porous drum 12 external to open container 15 (i.e., bottom section of water reservoir 13 is made round corresponding to roundness of revolving porous drum 12), without contacting it. This configuration drives significant portions of the streams of ambient air introduced into apparatus 10 by fan 14 through revolving porous drum 12. Water reservoir 13 communicates with open container 15 by means of pipe system 16 adapted for controllably discharging volumes of water from water reservoir 13 into open container by means of valve 16v (shown in FIG. 4). Since water reservoir 18 is mounted above open container 15 water can be streamed through pipe system 16 by force of gravity. Water reservoir 13 may be filled with water via opening 13o provided at its upper wall, wherein said opening 13o may be accessed via a respective opening provided in housing 18, by removing the sealing lid 13c.

Revolving porous drum 12 is preferably made from a spongy or fibrous material, such as, but not limited to, polyethylene, polypropylene, foamed polystyrene, polybutylene, or spongy metals (e.g., austenitic stainless steel). The diameter of revolving porous drum 12 may generally be in range of 40 to 55 cm, and its thickness may generally be in range of 2 to 4 cm. The rate of ambient air introduced into apparatus 10 may generally be in the range 100 to 200 m³/hr.

In a preferred embodiment of the invention open container 15 comprises a heating element 17, preferably mounted on its bottom wall, for heating the brine solution maintained thereinside. Heating element 17 is preferably an electrical heating element capable of producing heat powers of about 100 to 300 WAT, employed for heating the brine solution and increase vapor pressure whenever there is a need to increase the humidity in the stream of treated air discharged from apparatus 10. Heating element 17 may be further employed for reducing the level of brine solution in open container 15, whenever it is determined that the level of brine has been increased due to absorption of moisture from the stream of ambient air passed through apparatus 10, which may result in an overflow of the solution.

Open container 15 may further comprise one or more level determining means 19 (e.g., electrodes) for providing indications responsive to the level of the brine solution maintained thereinside, and RedOx electrodes (reduction-oxidation electrodes—not shown) for providing indications regarding the activity of the brine solution. Removable waist disposal vessel 15d is preferably attached to an opening 15o provided in the bottom wall of open container 15 for receiving waist precipitants obtained thereinside. The bottom wall of open container 15 is preferably curved for facilitating the drainage of the precipitants into opening 15o.

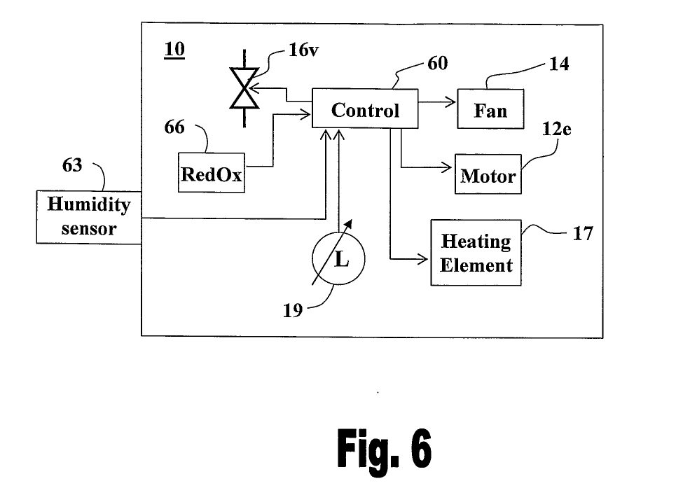

With reference to FIG. 6, the operation of apparatus 10 is preferably managed by means of a control unit 60 (e.g., a programmable microcontroller such as DSP33FJ256 of Microchip Technology Inc), electrically linked to fan 14, electrical motor 12e, to heating element 17, to level determining means 19, to valve 16v, to RedOx electrodes 66, and to a humidity sensor 63 (e.g., SHT1x or SHT7x of Sensirion). Humidity sensor 63 may be placed on housing 18 of apparatus 10, or inside housing 18 near one of the air inlet openings, or nearby (e.g., on a nearby wall), such that it is brought in contact with the ambient air. Fan 14 and electrical motor 12e are preferably adapted to operate responsive to control signals received from control unit 60, which is preferably also capable of setting a desired speed of rotations of said fan 14 and electrical motor 12e.

For example, control unit 60 may be adapted to increase the speed of rotations of electrical motor 12e (i.e., for increasing the speed of rotations of drum 12), or of electrical motor 14e (for increasing the rate of ambient air introduced into the device), responsive to readings received from the RedOx electrodes indicating low RedOx levels (e.g., less then 200 mV), for increasing the oxidation levels in the brine. Additionally or alternatively, the apparatus may further comprise an automatic whisk or mixer device installed in the container (not shown), and/or an air pump (not shown) adapted to introduce air bubbles into the brine solution, for increasing the oxidation levels of the solution.

The control unit may be adapted to increase the vapor pressure by activating the heating element 17 for discharging vapors with the stream of treated air discharged from apparatus 10, responsive to readings received from level determining means 19 indicating that the level of the brine solution in open container 15 exceeds an acceptable level (e.g., due to absorption of moisture from the ambient air streamed through apparatus 10).

Control unit 60 may be further adapted to alter the state of valve 16v according to indications received from level determining means 19 for streaming water from water reservoir 13, whenever the level of the brine solution in open container 15 is below an acceptable level. Water reservoir 13 preferably also comprises level determining means 13e (shown in FIG. 2), which may be also linked to control unit 60 for allowing it to issue alerts whenever the level of water maintained therein is too low. For this purpose control unit 60 may be linked to speakers, LEDs and/or display means, or other visual/audio output means, suitable for issuing alert indications. Similarly, control unit 60 may be adapted to issue alert indications responsive to RedOx readings indicating too low oxidation levels (e.g., less than 100 mV). A turbidity sensing means (e.g., photodiode—not shown) linked to the control unit may be installed inside open container 15, or waist disposal vessel 15d, near opening 15o, to allow control until 60 to issue visual/audio indications to the operator for replacing the waist disposal vessel 15d whenever it is filled with precipitants.

As described hereinabove, apparatus 10 may be used as a humidifier by employing heating element 17 for heating the brine solution in open container 15. This functionality is preferably managed responsive to indicating signals received by control unit 60 from humidity sensor 63. For example, control unit 60 may be adapted to activate heating element 17 whenever it is determined according to signals received from humidity sensor 63 that the air humidity is less than 50%. Furthermore, control unit 60 may be further adapted to change the state of valve 16v before, or during, activation of heating element 17, for streaming a volume of water from water reservoir 13 into open container 15.

A suitable set-up for measuring the Redox potential of the brine solution comprises a measuring electrode made of an inert metal or alloy (a platinum electrode) and a reference electrode (such as Ag/AgCl or calomel). Suitable electrodes are commercially available. The Redox potentials reported herein are measured using Pt/Ag/AgCl electrodes, thus indicating the electrochemical potential which is developed between Pt electrode exposed to the brine and a standard silver-silver chloride electrode.

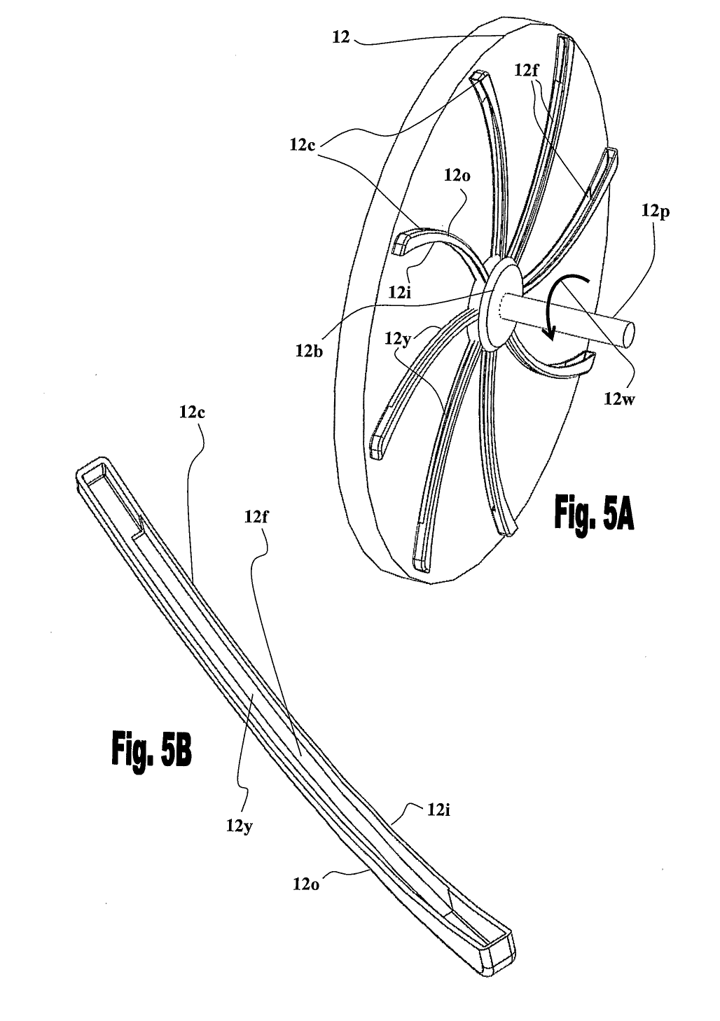

In one optional embodiment of the invention revolving porous drum 12 may optionally include liquid collecting means for increasing the amount of brine solution contained in its pores during operation. FIG. 5A shows an optional embodiment of revolving porous drum 12 comprising liquid collecting means 12c attached to one of its sides. Liquid collecting means 12c are preferably arced shaped elements attached on revolving porous drum 12 to a round base 12b attached over rotatable shaft 12p. A number of (e.g., about 6 to 10) collecting means 12c are preferably evenly distributed with more or less equal angles therebetween forming a structure similar to that of a wind mill. In this configuration, revolving porous drum 12 is rotated in the direction (indicated by arrow 12w) which introduces the inner arcs 12i of collecting means 12c first into open container.

As best seen in FIG. 5B, showing a perspective view of liquid collecting means 12c, a canal (ditch) 12f is formed in collecting means 12c, said canal 12f is formed in the side of inner arc 12i of collecting means 12c. An elongated slot 12y, communicating with canal 12f, is formed in the outer arc 12o of collecting means 12c, more or less centrally along its length. Similar slot (12o) may be may be also provided in one, or both, sides of collecting means 12c. Advantageously, elongated slot 12y is formed near the side of collecting means 12c attached to revolving porous drum 12, such that the brine solution obtained in canal 12f whenever it is rotated through the brine solution maintained in open container 15 is discharged from canal 12f through elongated slot 12y over a surface area of revolving porous drum 12, when said collecting means 12c is rotated out of the brine solution.

Experiments carried out with an apparatus of the invention using a spongy drum having a diameter of about 50 cm and thickness of about 2.5 cm, and a dead sea brine, showed that it is capable of reducing the amount of contaminants in the air by more than %50.

It is noted that the apparatus of the invention may be used in a wide range of applications requiring treatment of contaminated air. For example, the invention may be used for treating air in hospitals departments and particularly patients' rooms, in elderly care units (retirement houses) refrigerating facilities (devices and rooms), healthcare facilities, kinder gardens, and private houses. The invention may be also a benefit for people suffering from breathing difficulties (e.g., asthma) and those suffering from airborne allergens.

The above examples and description have of course been provided only for the purpose of illustration, and are not intended to limit the invention in any way. As will be appreciated by the skilled person, the invention can be carried out in a great variety of ways, employing more than one technique from those described above, all without exceeding the scope of the invention.

1. An apparatus for treating indoor air, the apparatus comprising a container suitable for holding a brine solution thereinside, a porous medium rotatably mounted on, or in, said container such that a section thereof is capable of being immersed in the brine solution held inside it, rotating means mechanically coupled to said porous medium, and air streaming means capable of directing a stream of air to a surface area of said porous medium.

2. The apparatus according to claim 1 further comprising humidity sensing means and means for monitoring and adjusting the temperature of a solution maintained inside the container responsive to indications received from said humidity sensing means.

3. The apparatus according to claim 1 further comprising a reservoir suitable for holding a volume of water, wherein said reservoir is in fluid communication with said container, and means for controlling water flow from said reservoir.

4. The apparatus according to claim 1 further comprising level determining means installed inside the container.

5. The apparatus according to claim 1 further comprising means for measuring a Redox potential installed inside the container.

6. The apparatus according to claim 1 wherein the porous medium is a rotating drum.

7. The apparatus according to claim 2 comprising a heating element and a temperature sensor installed inside the container, and control means adapted to activate said heating element in response to signals received from the humidity sensing means and/or said temperature sensor.

8. The apparatus according to claim 3, wherein the control means is further adapted to control the flow of water from the reservoir responsive to signal indications received from the level determining means.

9. The apparatus according to claim 5 further comprising means for adjusting the activity of the solution responsive to signal indications received from the Redox potential measuring means.

10. The apparatus according to claim 9 comprising an automated whisk or mixer device installed in the container.

11. The apparatus according to claim 9 comprising an air pump capable of introducing air bubbles into a solution held in the container.

12. The apparatus according to claim 1 wherein the porous medium comprises one or more elongated members formed in, or attached on, one or both of its sides, wherein each of said elongated members comprises an elongated ditch adapted to collect brine solution in it, and an elongated slit provided in the base of said elongated ditch and adapted to discharge brine solution therefrom.

13. A method for treating air comprising rotating a porous drum in a brine solution and forcing a stream of air to pass through a section of said drum which extends from said brine solution.

14. The method according to claim 13 further comprising adjusting the temperature of the brine solution for adding moisture to the treated air.

15. The method according to claim 13 further comprising periodically or continuously measuring the Redox potential of the brine solution, and wherever needed, adjusting its Redox potential by increasing the speed of the stream of air and/or by increasing the speed of rotation of the porous drum.

16. The method according to claim 13 further comprising periodically or continuously measuring the Redox potential of the brine solution, and wherever needed, adjusting its Redox potential by means of an automated whisk or mixer device installed in the container and adapted to introduce air bubbles into the solution.

17. The method according to claim 13 further comprising periodically or continuously measuring the Redox potential of the brine solution, and wherever needed, adjusting its Redox potential by means of an air pump capable of introducing air bubbles into the solution.
